(19) United States
(12) Patent Application Publication
(10) Pub. No.: US 2025/0161317 A1
(43) Pub. Date: May 22, 2025

(54) MOLIDUSTAT FORMULATIONS AND METHODS OF USE FOR TREATMENT OF ANEMIA IN CATS

(71) Applicants: ELANCO US INC., Greenfield, IN (US); ELANCO ANIMAL HEALTH GMBH, Monheim am Rhein (DE)

(72) Inventors: Gerald BEDDIES, Leverkusen (DE); Annette BOEGEL, Leichlingen (DE); Samuel CHARLES, Leverkusen (DE); Kristine FRAATZ, Leverkusen (DE); Stefan HOFMANN, Langenfeld (DE); Ricarda HUESKEN, Leverkusen (DE); Simone Marlene PUTZKE, Leverkusen (DE); Franziska SCHMIDT, Duesseldorf (DE); Anne-Katrin Laura ORLOVIUS, Leverkusen (DE)

(21) Appl. No.: 18/730,668

(22) PCT Filed: Sep. 29, 2022
(86) PCT No.: PCT/US2022/045207

§ 371 Date: Jul. 19, 2024

Related U.S. Application Data
(60) Provisional application No. 63/301,881, filed on Jan. 21, 2022.

(51) Int. Cl. A61K 31/5377; A61K 9/00; A61K 9/10; A61K 9/14; A61K 47/44; A61P 7/06; C12N 9/99

(52) U.S. Cl. CPC A61K 31/5377; A61K 9/0053; A61K 9/10; A61K 9/14; A61K 47/44; A61P 7/06; C12N 9/99

(57) ABSTRACT

Pharmaceutical compositions comprising molidustat and an oil for the treatment of anemia in cats and related methods of use are described.

### CROSS-REFERENCE TO RELATED APPLICATION

This patent application is an international patent application which claims priority to U.S. Provisional Application No. 63/301,881 filed on Jan. 21, 2022, the disclosure of which is incorporated herein in its entirety.

### BACKGROUND OF THE INVENTION

Anemia (anaemia) associated with chronic kidney disease (CKD) in cats is mostly caused by decreased erythropoietin (EPO) production in the kidney. Production of EPO is controlled by the hypoxia inducible factor (HIF). Higher levels of HIF increase EPO production, which occurs in low oxygen conditions. In CKD, the reduced metabolic activity of the failing kidney leads to a relative renal hyperoxia and therefore EPO production is reduced in spite of a preserved EPO control mechanism. Consequently, cats with chronic kidney disease (CKD) cannot produce sufficient EPO to maintain normal red blood cell levels. Accordingly, renal anemia is a common and serious complication of CKD that worsens with disease progression. There remains a need for compounds that stimulate EPO production and methods for treating or managing of anemia due to CKD. There further remains a need for stable formulations that are palatable and provide for improved delivery to cats.

### BRIEF SUMMARY OF THE INVENTION

This disclosure provides for various compositions comprising a hypoxia-inducible factor-prolyl hydroxylase (HIF-PH) inhibitor (e.g., molidustat or salt thereof) that increases endogenous erythropoietin production as well as methods of preparing and using such compositions.

For example, pharmaceutical compositions are provided herein.

Compositions described herein (e.g., pharmaceutical compositions) can comprise a hypoxia-inducible factor prolyl hydroxylase inhibitor and an oil. The hypoxia-inducible factor prolyl hydroxylase inhibitor can comprise a compound of Formula (I)

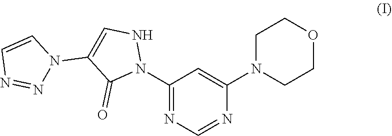 [SMILES: O=c1c(-n2ccnn2)c[nH]n1-c1cc(N2CCOCC2)ncn1]

or a salt, stereoisomer, tautomer, or N-oxide thereof.

The compound of Formula I can be in the form a salt having the Formula (II)

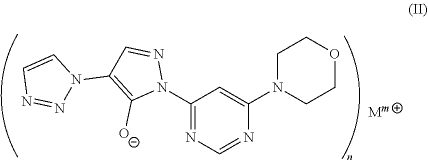

wherein M is lithium, sodium, potassium, calcium, magnesium, barium, manganese, copper, silver, zinc, iron, ammonium, or a substituted ammonium in which one to four of the hydrogen atoms are replaced by C₁-C₄-alkyl; m denotes the respective positive charge of the cation, being 1, 2, or 3, preferably 1; and n denotes the respective stoichiometric amount of the counter anion and is 1, 2, or 3, preferably 1; wherein n equals m so that the salt having the formula (II) is uncharged.

The hypoxia-inducible factor prolyl hydroxylase inhibitor can comprise a compound of Formula (IIA):

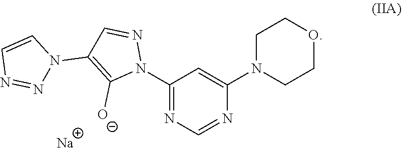 [SMILES: [O-]c1c(-n2ccnn2)cnn1-c1cc(N2CCOCC2)ncn1 ; [Na+]]

The hypoxia-inducible factor prolyl hydroxylase inhibitor can comprise a sodium salt. The hypoxia-inducible factor prolyl hydroxylase inhibitor can comprise or consist of molidustat. The hypoxia-inducible factor prolyl hydroxylase inhibitor can comprise or consist of molidustat sodium.

The pharmaceutical compositions can comprise micronized particles comprising the hypoxia-inducible factor prolyl hydroxylase inhibitor. The micronized particles can be characterized by a D90 that is about 70 μm or less, about 60 μm or less, about 50 μm or less, about 40 μm or less, or about 30 μm or less. The micronized particles can be characterized by a D90 that is from about 20 μm to about 70 μm, from about 20 μm to about 60 μm, from about 20 μm to about 50 μm, from about 20 μm to about 40 μm, or from about 20 μm to about 30 μm. The micronized particles can be characterized by a D10 that is from about 0.1 μm to about 10 μm, from about 0.2 μm to about 10 μm, or from about 0.2 to about 5 μm. The micronized particles can be characterized by a D50 that is from about 1 μm to about 20 μm, about 1 μm to about 15 μm, about 1 μm to about 10 μm, from about 5 μm to about 20 μm, from about 5 μm to about 15 μm, from about 5 μm to about 10 μm, from about 10 to about 20 μm, or from about 10 to about 15 μm. The micronized particles can be characterized by a substantially monomodal particle size distribution.

In pharmaceutical compositions as described herein, the hypoxia-inducible factor prolyl hydroxylase inhibitor (e.g., molidustat or molidustat sodium) concentration can be from about 1.0% and 20% (m/v). The hypoxia-inducible factor prolyl hydroxylase inhibitor concentration can be from about 1.0% and 10% (m/v). The hypoxia-inducible factor prolyl hydroxylase inhibitor concentration can be from about 1.0% and 5.0% (m/v). The hypoxia-inducible factor prolyl hydroxylase inhibitor concentration can be about 1.0%, 1.25%, 1.50%, 1.75%, 2.0%, 2.25%, 2.50%, 2.75%, 3.0%, 3.25%, 3.50%, 3.75%, 4.0%, 4.25%, 4.50%, 4.75%, or 5.0% (m/v) or a concentration within the bounds of any of these percentages. The hypoxia-inducible factor prolyl hydroxylase inhibitor concentration can be from about 1.5% and 2.5% (m/v), 2.0% and 4.5% (m/v), 2.0% and 3.0% (m/v), 1.0% and 3.0% (m/v), or 2.0% and 5.0% (m/v). For example, the hypoxia-inducible factor prolyl hydroxylase inhibitor concentration can be about 2.5% (m/v).

The pharmaceutical compositions described herein can comprise an oil that can comprise at least one selected from the group consisting of almond oil, apricot kernel oil, canola oil, castor oil, coconut oil, cottonseed oil, flaxseed oil, grape oil, hemp oil, maize oil, olive oil, palm oil, peanut oil, sesame seed oil, soya oil, sunflower oil, thistle oil, canola oil, rice bran oil, wheat germ oil, and a mixture thereof.

The pharmaceutical compositions described herein can comprise an oil that can comprise at least one selected from the group consisting of modified almond oil, modified apricot kernel oil, modified canola oil, modified castor oil, modified coconut oil, modified cottonseed oil, modified flaxseed oil, modified grape oil, modified hemp oil, modified maize oil, modified olive oil, modified palm oil, modified peanut oil, modified sesame seed oil, modified soya oil, modified sunflower oil, modified thistle oil, modified rapeseed oil, modified rice bran oil, modified wheat germ oil, and a mixture thereof, wherein the modification is obtained by alcoholysis, preferably with glycerol, propylene glycol, or low molecular polyethylene glycol. The oil can comprise sunflower oil. The oil can comprise modified maize oil.

The pharmaceutical compositions described herein can further comprise a fish oil. The composition can further comprise at least one fish oil selected from the group consisting of salmon oil, cod-liver oil, and a mixture thereof. The fish oil can be in an amount between about 0.01% to 5% (w/w). The fish oil can be in an amount between about 0.01% to 1.5% (w/w). The fish oil can be in an amount 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2%, 2.5%, 3%, 3.5%, 4%, 4.5%, or 5% (w/w) or a concentration within the bounds of any of these percentages.

The pharmaceutical compositions described herein can further comprise a thickener. The thickener can comprise a glycerol ester with C₁₂-C₂₄ fatty acids. The glycerol ester can be a monoester, a diester, a triester, or a mixture thereof. The thickener can comprise glycerol dibehenate. The thickener can be in an amount of between about 0.1% and 10% (w/w). The thickener can be in an amount between about 0.1% and 8% (w/w). The thickener can be in an amount between about 0.5% and 5% (w/w). The thickener can be in an amount between about 0.5% and 2.5% (w/w). The thickener can be in an amount of about 0.1%, 0.2%, 0.3%, 0.4%, 0.5%, 0.6%, 0.7%, 0.8%, 0.9%, 1.0%, 1%, 2%, 3%, 4%, 5%, 6%, 7%, 8%. 9%, or 10% (w/w) or a concentration within the bounds of any of these percentages. The thickener can be in an amount of about 1.0% (w/w).

The pharmaceutical compositions described herein can further comprise an antioxidant. For example, compositions described herein can further comprise at least one antioxidant selected from the group consisting of ascorbyl palmitate, butylhydroxytoluene, butylhydroxyanisole, citric acid, lecithin, propyl gallate, tocopherol, and a combination thereof. The antioxidant can be in an amount between about 0.01% to 2% (w/w). The antioxidant can be in an amount between about 0.01% to 1.5% (w/w). The antioxidant can be in an amount 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, or 2% (w/w) or a concentration within the bounds of any of these percentages.

The pharmaceutical compositions described herein can further comprise a preservative. For example, the compositions described herein can further comprise at least one preservative selected from the group consisting of ethanol, propylene glycol, butanol, chlorobutanol, benzoic acid, sorbic acid, para-hydroxybenzoic esters, and a combination thereof. The preservative can be in an amount between about 0.01% to 2% (w/w). The preservative can be in an amount between about 0.01% to 1.5% (w/w). The preservative can be in an amount 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, or 2% (w/w) or a concentration within the bounds of any of these percentages.

The pharmaceutical compositions can comprise a hypoxia-inducible factor prolyl hydroxylase inhibitor described herein in an amount of from 0.1% to 20%, optionally from 0.5% to 10% (w/w); an oil in an amount of from 50% to 99.8%, optionally from 70% to 98.97% (w/w); optionally a fish oil in an amount of from 0.01% to 5%, optionally from 0.01% to 1.5% (w/w); optionally a thickener in an amount of from 0.1% to 10%, optionally from 0.5% to 5% (w/w); optionally an antioxidant in an amount of from 0.01% to 2%, optionally from 0.01% to 1.5% (w/w), and optionally a preservative in an amount of from 0.01% to 2%, optionally from 0.01% to 1.5% (w/w).

The pharmaceutical compositions described herein can be formulated for oral administration, sublingual/buccal administration, or a combination thereof. For example, the compositions described herein can be formulated for oral administration.

The pharmaceutical compositions described herein can be a suspension, emulsion, slurry, dispersion, or solution. For example, the compositions described herein can be a suspension.

The pharmaceutical compositions described herein can further comprise a pharmaceutically acceptable carrier, excipient, lubricant, emulsifier, stabilizer, solvent, diluent, buffer, surfactant, or a combination thereof.

The pharmaceutical compositions described herein can be used for use in the manufacture of a medicament for treating anemia (e.g., for use in the manufacture of a medicament for treating anemia associated with chronic kidney disease (CKD)).

The pharmaceutical compositions described herein can be for use in the treatment of anemia. The anemia can be non-regenerative anemia. The anemia can be iron-deficiency anemia, pernicious anemia, aplastic anemia, chemotherapy-induced anemia (CIA), immune mediated hemolytic anemia (IMHA), or hemolytic anemia. The anemia can be associated with chronic kidney disease (CKD).

As noted, various methods of use are also provided herein. For example, a method for increasing erythropoietin can comprise administering a pharmaceutical composition described herein to a subject in need thereof. Further, a method for treating anemia can comprise administering the pharmaceutical composition described herein to a subject in need thereof. As noted, the anemia can be non-regenerative anemia. The anemia can be iron-deficiency anemia, pernicious anemia, aplastic anemia, chemotherapy-induced anemia (CIA), immune mediated hemolytic anemia (IMHA), or hemolytic anemia. The anemia can be associated with chronic kidney disease (CKD).

In the methods described herein, the pharmaceutical composition can be administered once daily. The pharmaceutical composition can be administered once daily for at least 28 consecutive days. The pharmaceutical composition can be administered once daily intermittently following the 28 consecutive days of administration. The pharmaceutical composition is not administered at least 3 days, at least 4 days, at least 5 days, at least 6 days, at least 7 days, at least 8 days, at least 9 days, at least 10 days, or at least 14 days following the 28 consecutive days of administration.

In the methods described herein, the subject in need thereof can be a mammal. Further, the subject can be a cat.

In the methods described herein, the pharmaceutical composition can be administered orally. The pharmaceutical composition described herein can be administered at sufficient dose to provide a maximum plasma concentration (Cmax) of the hypoxia-inducible factor prolyl hydroxylase inhibitor of about 0.5 mg/L or greater, about 1 mg/L or greater, 1.5 mg/l or greater, 2 mg/L or greater, 2.5 mg/L or greater, 3 mg/L or greater, or from about 0.5 mg/L to about 5 mg/L.

### BRIEF DESCRIPTION OF THE DRAWINGS

FIG. 1 depicts the effect of different dosages and formulations of molidustat sodium on plasma hematocrit levels, the mean (±standard deviation) hematocrit (HCT) over the course of 98 days. Treatment was ceased on SD 15 (groups treated with 10% suspensions) and SD 23 (group treated with 5% suspension), respectively.

FIG. 2 depicts the effect of different dosages and formulations of molidustat sodium on plasma hematocrit levels. The time course of the group mean±SD hematocrit are presented for the 28 days.

FIG. 3 depicts the mean (±standard deviation) plasma concentration of erythropoietin on Study Day 0 and Study Day 7. The molidustat sodium oily suspension was administered daily

FIG. 4 presents a particle size distribution histogram of molidustat sodium oily suspension formulated with micronized molidustat.

FIG. 5 presents a particle size distribution histogram of molidustat sodium oily suspension formulated with non-micronized molidustat.

FIG. 6 depicts a graph of the sedimentation analysis of the micronized formulation (circles) and the unmicronized formulation (triangles) over 48 h time span.

### DETAILED DESCRIPTION OF THE INVENTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs. Preferred methods and compositions are described, although any methods and compositions similar or equivalent to those described herein can be used in the practice or testing of the present invention.

### Treatment of Anemia Using Hypoxia-Inducible Factor Prolyl Hydroxylase Inhibitor (HIF-PHI)

Anemia, optionally anaemia associated with chronic kidney disease, may be treated by administration of a hypoxia-inducible factor prolyl hydroxylase inhibitor (HIF-PHI). These inhibitors are members of a class of drugs that act by inhibiting prolyl hydroxylase which is a decisive factor in the breakdown of the hypoxia-inducible factor (HIF) under normoxic conditions. The hypoxia-inducible factor prolyl hydroxylase inhibitor or a pharmaceutical composition comprising the inhibitor may be used for the treatment of cats suffering from non-regenerative anemia associated with chronic kidney disease (CKD). For example, the hypoxia-inducible factor prolyl hydroxylase inhibitor may be formulated, optionally as a sodium salt, in a 2.5% (m/v) oily suspension and used for once daily oral dosing at a rate of 5 mg HIF-PHI per kg body weight for the treatment of anemia in cats (e.g., anemia associated with CKD).

The hypoxia-inducible factor prolyl hydroxylase inhibitor preferably comprises a compound of Formula (I)

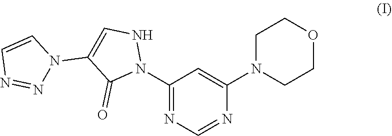 [SMILES: O=c1c(-n2ccnn2)c[nH]n1-c1cc(N2CCOCC2)ncn1]

or a salt, stereoisomer, tautomer, or N-oxide thereof. The compound of Formula (I) is also referred to as molidustat.

The hypoxia-inducible factor prolyl hydroxylase inhibitor may be a compound of Formula (I), which is in the form of a salt having the Formula (II)

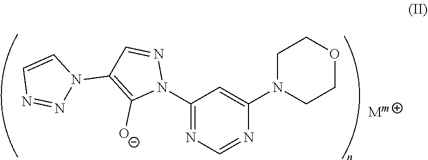

wherein
M is selected from the group consisting of lithium, sodium, potassium, calcium, magnesium, barium, manganese, copper, silver, zinc, iron, ammonium, and substituted ammonium in which one to four of the hydrogen atoms are replaced by C₁-C₄-alkyl, and preferably M is sodium; m denotes the respective positive charge of the cation, being 1, 2, or 3, preferably 1; and n denotes the respective stoichiometric amount of the counter anion and is 1, 2, or 3, preferably 1; wherein n equals m so that the salt having the Formula (II) is uncharged.

The hypoxia-inducible factor prolyl hydroxylase inhibitor may be in the form of the sodium salt of Formula (IIA)

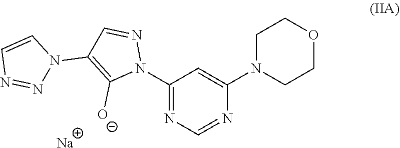 [SMILES: [O-]c1c(-n2ccnn2)cnn1-c1cc(N2CCOCC2)ncn1 ; [Na+]]

which is also known as sodium 1-[6-(morpholin-4-yl)pyrimidin-4-yl]-4-(1H-1,2,3-triazol-1-yl)-1H-pyrazol-5-olate.

The hypoxia-inducible factor prolyl hydroxylase inhibitor may be in the form of the potassium or ammonium salt of Formula (II), which is also known as potassium 1-[6-(morpholin-4-yl)pyrimidin-4-yl]-4-(1H-1,2,3-triazol-1-yl)-1H-pyrazol-5-olate or ammonium 1-[6-(morpholin-4-yl)pyrimidin-4-yl]-4-(1H-1,2,3-triazol-1-yl)-1H-pyrazol-5-olate.

The hypoxia-inducible factor prolyl hydroxylase inhibitor may be molidustat. For example, molidustat may be formulated as a sodium salt in a 2.5% (m/v) oily suspension and used for once daily oral dosing at a rate of 5 mg molidustat sodium per kg body weight for the treatment of anemia in cats. Molidustat, 2-[6-(morpholin-4-yl)pyrimidin-4-yl]-4-(1H-1,2,3-triazol-1-yl)-2,3-dihydro-1H-pyrazol-3-one, may be used in methods for treating anemia associated with chronic kidney disease in cats as described herein.

### Methods of Treatment

The disclosure further provides methods for treating or preventing anemia. The method comprises administering to a mammal a therapeutically or prophylactically a pharmaceutical composition comprising an effective amount of a hypoxia-inducible factor prolyl hydroxylase inhibitor. The anemia to be treated or prevented may be associated with chronic kidney disease.

A method for treating or preventing anemia, optionally associated with chronic kidney disease, may comprise administering to a mammal thereof a pharmaceutical composition comprising a therapeutically or prophylactically effective amount of a hypoxia-inducible factor prolyl hydroxylase inhibitor (e.g., molidustat or salt thereof such as molidustat sodium). “Therapeutically effective amount,” as used herein, refers broadly to an amount of a compound disclosed herein, that is effective for preventing, ameliorating, treating or delaying the onset of a disease or condition. The expression “prophylactically effective amount” refers to an amount of a compound disclosed herein, that is effective for inhibiting the onset or progression of a disorder.

A method for treating anemia, optionally associated with chronic kidney disease, may comprise administering to a mammal thereof a pharmaceutical composition comprising an effective amount of a hypoxia-inducible factor prolyl hydroxylase inhibitor.

A method of treating or preventing anemia, optionally non-regenerative anemia, may comprise administering to a mammal thereof a pharmaceutically composition comprising a therapeutically or prophylactically effective amount of a hypoxia-inducible factor prolyl hydroxylase inhibitor.

A method of treating anemia, optionally non-regenerative anemia, may comprise administering to a mammal thereof a pharmaceutically composition comprising an effective amount of a hypoxia-inducible factor prolyl hydroxylase inhibitor.

The anemia may be non-regenerative anemia. The anemia can be iron-deficiency anemia, pernicious anemia, aplastic anemia, hemolytic anemia, anemia associated with inflammatory disease, chemotherapy-induced anemia (CIA), or immune mediated hemolytic anemia (IMHA). The anemia may be associated with chronic kidney disease (CKD).

A method for increasing erythropoietin can comprise administering the pharmaceutical composition described herein to a subject in need thereof.

The pharmaceutical composition described herein can be administered once daily. The pharmaceutical composition described herein can be administered once daily for at least 28 consecutive days. The pharmaceutical composition described herein can be administered once daily for 1, 2, 3, 4, 5, 6, 7, 8, 9, 10, 11, 12, 13, 14, 15, 16, 17, 18, 19, 20, 21, 22, 23, 24, 25, 26, 27, or 28 days. The pharmaceutical composition described herein can be administered once daily for between about 1-28 days. The pharmaceutical composition described herein can be administered once daily for between about 1-7 days, 1-14 days, 1-21 days, 3-21 days, 16-28 days, or 14-21 days.

The subject may be a mammal. For example, the mammal may be a cat.

The pharmaceutical composition described herein can be administered orally.

The effective amount of the hypoxia-inducible factor prolyl hydroxylase inhibitor may be about 5 mg/kg of body weight. For example, the effective amount of the hypoxia-inducible factor prolyl hydroxylase inhibitor may be between about 1 and 10 mg per kg of body weight. The effective amount of the hypoxia-inducible factor prolyl hydroxylase inhibitor may be between about 1, 2, 3, 4, 5, 6, 7, 8, 9, or 10 mg per kg of body weight.

The hypoxia-inducible factor prolyl hydroxylase inhibitor may be present in the pharmaceutical composition in an amount of between about 1.0% and 5.0% (m/v). The hypoxia-inducible factor prolyl hydroxylase inhibitor may be in an amount of about 1.0%, 1.25%, 1.50%, 1.75%, 2.0%, 2.25%, 2.50%, 2.75%, 3.0%, 3.25%, 3.50%, 3.75%, 4.0%, 4.25%, 4.50%, 4.75%, or 5.0% (m/v) or a concentration within the bounds of any of these percentages. The hypoxia-inducible factor prolyl hydroxylase inhibitor may be in an amount of between about 1.5% and 2.5% (m/v), 2.0% and 4.5% (m/v), 2.0% and 3.0% (m/v), 1.0% and 3.0% (m/v), or 2.0% and 5.0% (m/v). The hypoxia-inducible factor prolyl hydroxylase inhibitor may be in an amount of about 2.5%. The hypoxia-inducible factor prolyl hydroxylase inhibitor can be molidustat. The hypoxia-inducible factor prolyl hydroxylase inhibitor can be the compound of Formula (I), Formula (II), Formula (IIA), or a combination thereof.

The pharmaceutical composition may be formulated as a suspension, emulsion, slurry, dispersion, or solution. The pharmaceutical composition may be a suspension.

### Dosage

The pharmaceutical compositions described herein are administered to a subject in a manner known in the art. The dosage administered will be dependent upon the age, health, and weight of the recipient, kind of concurrent treatment, if any, frequency of treatment, and the nature of the effect desired.

The hypoxia-inducible factor prolyl hydroxylase inhibitor may be present in any suitable amount within the pharmaceutical compositions described herein. Those of skill in the art can readily determine suitable concentrations of compound to include in the pharmaceutical compositions depending on various factors including dosage and route of administration. Pharmaceutical compositions useful in the present invention can contain a quantity of a hypoxia-inducible factor prolyl hydroxylase inhibitor in an amount effective to treat or prevent the condition, disorder or disease of the subject being treated.

The hypoxia-inducible factor prolyl hydroxylase inhibitor may be present in the pharmaceutical composition in an amount of from about 1.0% and 5.0% (m/v). The hypoxia-inducible factor prolyl hydroxylase inhibitor may be in an amount of about 1.0%, 1.25%, 1.50%, 1.75%, 2.0%, 2.25%, 2.50%, 2.75%, 3.0%, 3.25%, 3.50%, 3.75%, 4.0%, 4.25%, 4.50%, 4.75%, or 5.0% (m/v) or a concentration within the bounds of any of these percentages. The hypoxia-inducible factor prolyl hydroxylase inhibitor may be in an amount of between about 1.5% and 2.5% (m/v), 2.0% and 4.5% (m/v), 2.0% and 3.0% (m/v), 1.0% and 3.0% (m/v), or 2.0% and 5.0% (m/v). The hypoxia-inducible factor prolyl hydroxylase inhibitor can be in an amount of about 2.5%.

The pharmaceutical compositions described herein may be administered in a single daily dose, or the total daily dosage may be administered in divided doses of two, three, or four times daily. Doses maybe administered for one week, one month, or over the course of several months, 3, 6, 9 or 12 months, or intervals known in the art and determined to be clinically relevant. Doses may be continued throughout the life of the subject or discontinues when clinical judgment warrants.

The daily dosage of the pharmaceutical compositions described herein may be varied over a wide range from about 1 to about 10 mg per subject, per day. The subject can be an animal. The subject can be mammal. The subject can be a cat. The range can be from about 1 mg/kg to 10 mg/kg of body weight per day. Additionally, the dosages may be about 0.5-20 mg/kg per day, about 1-10 mg/kg per day, about 2.5-10 mg/kg per day, about 5-10 mg/kg per day, or about 2.5 to 7.5 mg/kg per day. The daily dosage of the pharmaceutical compositions described herein can be about 1, 2, 3, 4, 5, 6, 7, 8, 9, or 10 mg/kg per subject, per day. In the case of other animals, the dose calculated for 1 kg may be administered as well.

As a non-limiting example, treatment of animals can be provided as a one time or periodic dosage of a pharmaceutical composition described herein may be 0.0001 to about 1,000 mg per subject, per day. The range may more particularly be from about 0.001 mg/kg to 10 mg/kg of body weight per day, about 0.1-100 mg, about 1.0-50 mg or about 1.0-20 mg per day for subject. Additionally, the dosages may be about 0.5-10 mg/kg per day, about 1.0-5.0 mg/kg per day, 5.0-10 mg/kg per day.

The dosage can also be an amount that achieve a serum concentration.

The pharmaceutical compositions of the present invention may be administered at least once a week over the course of several weeks. The pharmaceutical compositions may be administered at least once a week over several weeks to several months. The pharmaceutical compositions may be administered once a week over four to eight weeks. The pharmaceutical compositions may be administered once a week over four weeks. The pharmaceutical compositions may be administered once daily.

### Routes of Administration

Routes of administration and dosages of effective amounts of the pharmaceutical compositions comprising the compounds are also disclosed. The compounds of the present invention can be administered in combination with other pharmaceutical agents in a variety of protocols for effective treatment of disease.

The pharmaceutical compositions disclosed herein may be administered by the following routes, including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarenal, intraretinal, intraspinal, intrasynovial, intrathoracic, intrauterine, intravesical, bolus, vaginal, rectal, buccal, sublingual, intranasal, iontophoretic means, or transdermal means. The pharmaceutical compositions disclosed herein may be administered by injection. The pharmaceutical compositions disclosed herein may be administered orally.

### Subjects

The pharmaceutical compositions described herein can be administered to any animal that can experience the beneficial effects of the hypoxia-inducible factor prolyl hydroxylase inhibitor. Such animals include humans and non-humans such as pets and farm animals. Animals may include but are not limited to humans, cats, dogs, mice, rats, Guinea pigs, horses, donkeys, mules, sheep, cattle, goats, llamas, and hamsters. The animal may be a cat.

### Pharmaceutical Compositions

As noted, pharmaceutical compositions comprise at least one hypoxia-inducible factor prolyl hydroxylase inhibitor, especially comprises molidustat as described herein (i.e., a compound of Formula (I)) or a salt, stereoisomer, tautomer, or N-oxide thereof. The molidustat can be formulated as a salt. For example, the molidustat can be formulated as a sodium salt.

The pharmaceutical compositions described herein may further comprise at least one of any suitable auxiliaries including, but not limited to, diluents, binders, stabilizers, buffers, thickeners, antioxidants, salts, lipophilic solvents, surfactants, preservatives, adjuvants, or combinations thereof. Examples and methods of preparing such sterile solutions are well known in the art and can be found in well-known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the compound. “Pharmaceutically acceptable carrier,” as used herein, refers broadly to any and all solvents, dispersion media, coatings, antibacterial and antifungal agent, isotonic and absorption delaying agents for pharmaceutical active substances as are well known in the art. Except insofar as any conventional media or agent is incompatible with the compound, its use in the therapeutic compositions is contemplated. Supplementary compounds can also be incorporated into the compositions.

The pharmaceutical composition may comprise a surfactant. Suitable surfactants are amphiphilic compounds. Mono-, di-, or tri-esters of sorbitan with fatty acids, polyoxyethylated compounds, such as polyoxyethylene sorbitan fatty acid esters, polyoxyethlyene castor oil derivatives, and poloxamers, may be used as surfactants. Polyoxyethylated compounds, also referred to as polyethoxylated compounds, are prepared for example by reaction with ethylene oxide. They have one or more concatenated units of the formula —[O—CH₂CH₂]. Polyoxyethylated compounds which may be mentioned in particular are: nonionic amphiphilic polyoxyethylated compounds such as
poloxamers, preferably with molar masses of from 100 to 5000 g/mol, particularly preferably with molar masses of from 1000 to 3500 g/mol. Poloxamer is the international non-proprietary name for block copolymers of ethylene oxide and methyloxirane,
polyoxyethylene fatty acid glycerides, also called non-ionic emulsifiers, preferably for example glycerol polyethylene glycol ricinoleate,
polyoxyethylene sorbitan fatty acid esters, preferably for example polyoxyethylene 20 sorbitan monooleate,
polyoxyethylene fatty acids such as macrogol 15 hydroxystearate (=Solutol HS15, obtainable by reacting 15 mol of ethylene oxide and 1 mol of 12-hydroxystearic acid)
polyoxyethylene fatty alcohols such as hydroxypolyethoxydodecane.

Fatty acid or fatty alcohol stands in particular for the corresponding compounds having at least 6 carbon atoms and normally not more than 30 carbon atoms.

The pharmaceutical composition can comprise a thickener. The thickener may be in an amount of from 0.1% to 10% (w/w), from 0.1% to 8% (w/w), from 0.5% to 5% (w/w), or from 0.5% to 2.5% (w/w). The pharmaceutical composition can comprise thickener is in an amount of about 0.1%, 0.2%, 0.3%, 0.4%, 0.5%, 0.6%, 0.7%, 0.8%, 0.9%, 1.0%, 1%, 2%, 3%, 4%, 5%, 6%, 7%, 8%. 9%, or 10% (w/w) or a concentration within the bounds of any of these percentages. The pharmaceutical composition can comprise a thickener is in an amount of about 1.0% (w/w).

Suitable thickeners include but are not limited to cellulose derivatives, for example, methylcellulose, hydroxypropylcellulose, hydroxyethylcellulose, hydroxypropylmethylcellulose, carboxymethylcellulose, microcrystalline cellulose; bentonites, kaolin, pectin, starches, modified starch, waxes, agar, paraffins, gelatin, alginates, polyvinylpyrrolidone, crospovidone, cetyl alcohol, stearates such as, for example, magnesium stearate, zinc stearate or glyceryl stearate, saturated or unsaturated long-chain fatty acids (C₈-C₂₄, high molecular weight polyethylene glycols (e.g., polyethylene glycol 2000), glycerol ester, and combinations thereof.

The thickener can be a glycerol ester and is preferably a glycerol ester with C₁₂-C₂₄ fatty acids and/or is a monoester, a diester, a triester, or a mixture thereof. The thickener may be glycerol dibehenate. Glycerol dibehenate is also referred to as glyceryl dibehenate or glycerin dibehenate.

The pharmaceutical composition may comprise an antioxidant. The antioxidant can be in an amount of from 0.01% to 2% (w/w), from 0.01% to 1.5% (w/w), 0.5 to 2 wt. %, from 0.01% to 1.5% (w/w), from 0.001% to 1% (w/w), or from 0.01% to 0.3% (w/w). The pharmaceutical compositions described herein can comprise an antioxidant in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, or 2% (w/w) or a concentration within the bounds of any of these percentages.

Suitable antioxidants include but are not limited to ascorbyl palmitate, butylhydroxytoluene, butylhydroxyanisole, lecithins, sulfites (Na sulfite, Na metabisulfite), organic sulfides (cystine, cysteine, cysteamine, methionine, thioglycerol, thioglycolic acid, thiolactic acid), phenols (tocopherols, as well as vitamin E and vitamin E DPGS (d-alpha-tocopheryl polyethylene glycol 1000 succinate)), butylated hydroxyanisole, butylated hydroxytoluene, gallic acid (propyl, octyl, propyl gallate, and dodecyl gallate), organic acids (ascorbic acid, citric acid, tartaric acid, lactic acid) and salts and esters thereof may be mentioned. Preferably, antioxidants may be selected from the group consisting of ascorbyl palmitate, butylhydroxytoluene, butylhydroxyanisole, citric acid, lecithins, propyl gallate, and tocopherol.

The pharmaceutical composition can comprise an antioxidant selected from the group consisting of ascorbyl palmitate, butylhydroxytoluene, butylhydroxyanisole, citric acid, lecithins, propyl gallate, tocopherol, or a combination thereof.

The pharmaceutical compositions described herein can comprise a preservative. Suitable preservatives include but are not limited to carboxylic acids (sorbic acid, propionic acid, benzoic acid, lactic acid), phenols (cresols, p-hydroxybenzoic esters such as methylparaben, propylparaben), aliphatic alcohols (benzyl alcohol, ethanol, butanol), quaternary ammonium compounds (benzalkonium chloride, cetylpyridinium chloride). Preferably, preservatives may be ethanol, propylene glycol, butanol, chlorobutanol, benzoic acid, sorbic acid, and para-hydroxybenzoic esters. Methyl 4-hydroxybenzoate, ethyl 4-hydroxybenzoate, and propyl 4-hydroxybenzoate are also known as preferred para-hydroxybenzoic esters.

The pharmaceutical composition can comprise a preservative selected from the group consisting of ethanol, propylene glycol, butanol, chlorobutanol, benzoic acid, sorbic acid, para-hydroxybenzoic esters, and combinations thereof.

Pharmaceutical excipients and additives useful in the present invention can also include, but are not limited to, proteins, peptides, amino acids, lipids, and carbohydrates (e.g., sugars, including monosaccharides, di-, tri-, terra-, and oligosaccharides; derivatized sugars such as alditols, aldonic acids, esterified sugars; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, casein. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, aspartame, and combinations thereof.

Carbohydrate excipients suitable for use in the present invention include monosaccharides e.g., fructose, maltose, galactose, glucose, D-mannose, sorbose; disaccharides, e.g., lactose, sucrose, trehalose, cellobiose; polysaccharides, such as raffinose, melezitose, maltodextrins, dextrans, starches; and alditols, e.g., mannitol, xylitol, maltitol, lactitol, xylitol, sorbitol (glucitol), myoinositol and combinations thereof.

The pharmaceutical compositions described herein may further comprise coloring agents, emulsifying agents, surfactants, thickening agents, suspending agents, ethanol, chelators (e.g., EDTA), buffers (e.g., citrate buffer), flavoring, water, or combinations thereof.

Chelators such as EDTA and EGTA can optionally be added to the pharmaceutical compositions to reduce aggregation. These additives are particularly useful if a pump or plastic container is used to administer the pharmaceutical composition. The presence of pharmaceutically acceptable surfactant mitigates the propensity for the composition to aggregate.

The pharmaceutical compositions described herein may comprise an emulsifier, including, but are not limited to ethyl alcohol, isopropyl alcohol, ethyl carbonate, ethyl acetate, benzyl alcohol, benzyl benzoate, propylene glycol, 1,3-butylene glycol, dimethyl formamide, oils, glycerol, tetrahydrofurfuryl alcohol, polyethylene glycols and fatty acid esters of sorbitan, and mixtures thereof.

Additionally, the pharmaceutical compositions described herein can comprise polymeric excipients/additives such as polyvinylpyrrolidones, ficolls (a polymeric sugar), dextrates (e.g., cyclodextrins, such as hydroxypropyl-ß-cyclodextrin), polyethylene glycols, flavoring agents, anti-microbial agents, sweeteners, antioxidants, anti-static agents, surfactants (e.g., polysorbates such as “Tween® 20” and “Tween® 80”), lipids (e.g., phospholipids, fatty acids), steroids (e.g., cholesterol), and chelating agents (e.g., EDTA or EGTA). These and additional known pharmaceutical excipients and/or additives suitable for use in the pharmaceutical compositions described herein and are known in the art, e.g., as listed in REMINGTON: THE SCIENCE & PRACTICE OF PHARMACY (19th ed., Williams & Williams (1995)) and PHYSICIAN'S DESK REFERENCE (52nd ed., Medical Economics (1998)).

The present disclosure provides stable pharmaceutical compositions as well as preserved solutions and formulations containing a preservative, as well as multi-use preserved formulations suitable for pharmaceutical or veterinary use, comprising at least one compound disclosed herein in a pharmaceutically acceptable formulation.

The compounds disclosed herein can also be administered in the form of liposomes. As is known in the art, liposomes are generally derived from phospholipids or other lipid substances. Liposomes are formed by mono-or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the present invention, stabilizers, preservatives, excipients, or combinations thereof. The preferred lipids are the phospholipids and the phosphatidyl cholines (lecithins), both natural and synthetic. Methods to form liposomes are known in the art (see Prescott, ed., METH. CELL BIOL. 14:33 (1976)). Liposomes, methods of making and methods of use are described in U.S. Pat. No. 4,089,8091 (process for the preparation of liposomes), U.S. Pat. No. 4,233,871 (methods regarding biologically active materials in lipid vesicles), U.S. Pat. No. 4,438,052 (process for producing mixed miscelles), U.S. Pat. No. 4,485,054 (large multilamellar vesicles), U.S. Pat. No. 4,532,089 (giant-sized liposomes and methods thereof), U.S. Pat. No. 4,897,269 (liposomal drug delivery system), U.S. Pat. No. 5,820,880 (liposomal formulations).

The hypoxia-inducible factor prolyl hydroxylase inhibitor can be solubilized or suspended in a preconcentrate (before dilutions with a diluent), added to the preconcentrate prior to dilution, added to the diluted preconcentrate, or added to a diluent prior to mixing with the preconcentrate. The hypoxia-inducible factor prolyl hydroxylase inhibitor can also be co-administered as part of an independent dosage form, for therapeutic effect. Optionally, the hypoxia-inducible factor prolyl hydroxylase inhibitor can be present in a first, solubilized amount, and a second, non-solubilized (suspended) amount.

The pharmaceutical compositions described herein can be presented in unit-dose or multi-dose containers, sealed ampules and vials, and may be stored in a freeze-dried (lyophilized) condition requiring only the addition of the sterile liquid carrier, water for injections, immediately prior to use. Extemporaneous suspensions can be prepared from sterile powders, granules and tablets.

Acceptable liquid carriers for use in the pharmaceutical compositions, include, but are not limited to, vegetable oils, e.g., peanut oil, cotton seed oil, sesame oil or combinations thereof. The pharmaceutical compositions can be prepared by dissolving or suspending the hypoxia-inducible factor prolyl hydroxylase inhibitor in the liquid carrier such that the final formulation contains from about 0.5% to 5.0% (m/v).

For oral administration in the form of a tablet or capsule, hypoxia-inducible factor prolyl hydroxylase inhibitor can be combined with an oral, non-toxic pharmaceutically acceptable inert carrier such as ethanol, glycerol, water or combinations thereof. Moreover, when desired or necessary, suitable binders, lubricants, disintegrating agents, and coloring agents may also be incorporated into the pharmaceutical compositions. Suitable binders include, without limitation, starch; gelatin; natural sugars including but not limited to glucose or beta-lactose; corn sweeteners; natural and synthetic gums such as acacia, tragacanth, or sodium alginate, carboxymethylcellulose; polyethylene glycol; waxes, or combinations thereof. Lubricants used in these dosage forms include, without limitation, sodium oleate, sodium stearate, magnesium stearate, sodium benzoate, sodium acetate, sodium chloride, or combinations thereof. Disintegrators include, without limitation, starch, methyl cellulose, agar, bentonite, xanthan gum, or combinations thereof.

For oral administration, the pharmaceutical composition may not comprise a sweetener. Sweeteners include but are not limited to sucrose, fructose, sodium saccharin, sucralose (SPLENDA®), sorbitol, mannitol, aspartame, sodium cyclamate, and combinations thereof. The pharmaceutical compositions described herein may be substantially free of sweeteners. The pharmaceutical compositions described herein may not comprise flavoring agents, for example vanilla, anise, honey flavor, or a combination thereof.

The pharmaceutical compositions described herein formulated for oral administration can be combined with coloring agents, e.g., dye stuffs, natural coloring agents or pigments, in addition to the diluents such as water, glycerin and various combinations. Methods of preparing said pharmaceutical compositions can incorporate other suitable pharmaceutical excipients and their formulations as described in REMINGTON'S PHARMACEUTICAL SCIENCES, Martin, E. W., ed., Mack Publishing Company, 19th ed. (1995).

### Micronization

The composition can comprise micronized particles comprising the hypoxia-inducible factor prolyl hydroxylase inhibitor. The micronized particles are characterized by a D90 that is about 70 μm or less, about 60 μm or less, about 50 μm or less, about 40 μm or less, or about 30 μm or less. The micronized particles can be characterized by a D90 that is from about 20 μm to about 70 μm, from about 20 μm to about 60 μm, from about 20 μm to about 50 μm, from about 20 μm to about 40 μm, or from about 20 μm to about 30 μm. The micronized particles can be characterized by a D10 that is from about 0.1 μm to about 10 μm, from about 0.2 μm to about 10 μm, or from about 0.2 to about 5 μm. The micronized particles can be characterized by a D50 that is from about 1 μm to about 20 μm, about 1 μm to about 15 μm, about 1 μm to about 10 μm, from about 5 μm to about 20 μm, from about 5 μm to about 15 μm, from about 5 μm to about 10 μm, from about 10 to about 20 μm, or from about 10 to about 15 μm. The micronized particles can be characterized by a substantially monomodal particle size distribution.

The terms “D10” and “Dx10” describe the diameter of the measured particles where 10% of the distribution has a smaller particle size and 90% has a larger particle size by volume. The terms “D50” and “Dx50” describe the diameter of the particles where 50% of the distribution has a smaller and 50% has a larger particle size by volume. The terms “D90” and “Dx90” describe the diameter of the particles where 90% of the distribution has smaller and 10% has larger particle size by volume.

Micronization of the hypoxia-inducible factor prolyl hydroxylase inhibitor allows for greater suspension properties in oils. Micronization of the hypoxia-inducible factor prolyl hydroxylase inhibitor particles has been found to beneficially prevent agglomerization, provide for the particles to stay loosely flocculated in the suspension, reduce the rate of sedimentation, and permit easy re-homogenization throughout the shelf life of the product. For example, micronized hypoxia-inducible factor prolyl hydroxylase inhibitor particles stay in suspension longer than non-micronized particles, e.g., over six hours versus less than 1 hour. It has been surprisingly discovered that the micronized particles can stay in suspension for over 48 hours with no significant sedimentation. Accordingly, micronization of the hypoxia-inducible factor prolyl hydroxylase inhibitor provides for more consistent and predictable dosing of the hypoxia-inducible factor prolyl hydroxylase inhibitor.

The inventors found that the formulation of the micronized hypoxia-inducible factor prolyl hydroxylase inhibitor particles in sunflower oil using glycerl dibehenate as a thickener provided an unexpected improvement in the suspension. Without wishing to be bound to a particular theory, the inventors found that micronized hypoxia-inducible factor prolyl hydroxylase inhibitor particles formulated into an oleogel-like structure formed by sunflower-oil and glyceryl dibehenate. The gelling concept follows the concept of disordered structures. The thickener glyceryl dibehenate is a polar fat which is solid at room temperature, but which becomes plasticized in sunflower oil (vegetable oils in general) at temperatures above 40° C. Above this temperature, the binary system sunflower oil and glyceryl dibehenate forms a clear oily liquid. Upon cooling, the glyceryl dibehenate starts to recrystallize into small crystalline particles which form a network like structure in the sunflower oil. This situation is equivalent to the Bentonite structuring mechanism in aqueous systems. When the concentration of glyceryl dibehenate is high enough, an organogel is formed, where the three-dimensional network of partially flocculated crystals takes up the whole volume of the vehicle. When the concentration is lower, the particles form loose structures and flocs within the sunflower oil which sediment slowly over time. This sediment structure then provides the support for micronized hypoxia-inducible factor prolyl hydroxylase inhibitor particles settling within the glyceryl dibehenate network, in card house-like structure. The co-sediment of the micronized hypoxia-inducible factor prolyl hydroxylase inhibitor particles and glyceryl dibehenate is then easy to redisperse. The versatility of those disordered particulate organogels lies in the fact that no specific physicochemical interaction between the thickener and the supported the micronized hypoxia-inducible factor prolyl hydroxylase inhibitor particles is necessary.

The pharmacokinetic data derived for molidustat sodium oily suspension 2.5% show a high and almost complete bioavailability (approx. 80%) after oral administration in the target animal cat.

### Oily Suspensions

The pharmaceutical composition may be formulated as an oily suspension. The oil may be almond oil, apricot kernel oil, canola oil, castor oil, coconut oil, cottonseed oil, flaxseed oil, grape oil, hemp oil, maize oil, olive oil, palm oil, peanut oil, sesame seed oil, soya oil, sunflower oil, thistle oil, canola oil, rice bran oil, wheat germ oil, or mixtures thereof. The oil may be sunflower oil.

Maize oil (corn oil) may be obtained from seeds of Zea mays L. by expression or by extraction followed by an optional refining. Preferably, the maize oil comprises 8.6% to 16.5% (w/w) of palmitic acid, up to 3.3% (w/w) of stearic acid, 20% to 42.2% (w/w) of oleic acid, 39.4% to 65.6% (w/w) of linoleic acid, 0.5% to 1.5% (w/w) of arachidic acid, up to 0.5% (w/w) of eicosenoic acid, and up to 0.5% (w/w) of behenic acid, based on the total amount of fatty acids.

Sunflower oil may be obtained from seeds of Helianthus annuus by mechanical expression or by extraction followed by an optional refining. Preferably, sunflower oil comprises 4% to 9% (w/w) of palmitic acid, 1% to 7% (w/w) of stearic acid, 14% to 40% (w/w) of oleic acid, and 48% to 74% (w/w) of linoleic acid, based on the total amount of fatty acids.

Thistle oil (safflower oil) may be obtained from seeds of Carthamus tinctorius L. (type I) or from seeds of hybrids of Carthamus tinctorius L. (type II) by expression and/or extraction followed by an optional refining. Preferably, the thistle oil obtained from type I fraction comprises up to 0.2% (w/w) of saturated fatty acids of chain length less than C₁₄, up to 0.2% (w/w) of myristic acid, 4% to 10% (w/w) of palmitic acid, 1% to 5% (w/w) of stearic acid, 8% to 21% (w/w) of oleic acid, 68% to 83% (w/w) of linoleic acid, up to 0.5% (w/w) of linolenic acid, up to 0.5% (w/w) of arachidic acid, up to 0.5% (w/w) of eicosenoic acid, and up to 1% (w/w) of behenic acid, based on the total amount of fatty acids. Preferably, the thistle oil obtained from type II fraction comprises up to 0.2% (w/w) of saturated fatty acids of chain length less than C₁₄, up to 0.2% (w/w) of myristic acid, 3.6% to 6% (w/w) of palmitic acid, 1% to 5% (w/w) of stearic acid, 70% to 84% (w/w) of oleic acid, 7% to 23% (w/w) of linoleic acid, up to 0.5% (w/w) of linolenic acid, up to 1% (w/w) of arachidic acid, up to 1% (w/w) of eicosenoic acid, and up to 1.2% (w/w) of behenic acid, based on the total amount of fatty acids.

The pharmaceutical composition may comprise a modified oil, wherein the modification is obtained by alcoholysis, preferably with glycerol, propylene glycol, or low molecular polyethylene glycol. In this connection, it is to be understood that low molecular polyethylene glycol are defined as follows: H—(O—CH₂—CH₂)ₙ—OH, wherein n is selected from 1 to 5,preferably from 1 to 4, and, optionally, from 1 to 3 or from 1 to 2.

The modified oil may be modified almond oil, modified apricot kernel oil, modified canola oil, modified castor oil, modified coconut oil, modified cottonseed oil, modified flaxseed oil, modified grape oil, modified hemp oil, modified maize oil, modified olive oil, modified palm oil, modified peanut oil, modified sesame seed oil, modified soya oil, modified sunflower oil, modified thistle oil, modified rapeseed oil, modified rice bran oil, modified wheat germ oil, or mixtures thereof, wherein the modification is obtained by alcoholysis, preferably with glycerol, propylene glycol, or low molecular polyethylene glycol. In this connection, it is to be understood that low molecular polyethylene glycol are defined as follows: H—(O—CH₂—CH₂)ₙ—OH, wherein n is selected from 1 to 5, preferably from 1 to 4, and, optionally, from 1 to 3 or from 1 to 2.

Alcoholysis is an example of a solvolysis reaction, wherein the triglyceride reacts with an alcohol such as methanol or ethanol to give the methyl or ethyl esters of the fatty acid. Optionally, glycerol may be used as alcohol. This reaction is also known as a transesterification reaction due to the exchange of the alcohol fragments. The alcoholysis reaction is preferably followed by a winterization process to eliminate certain saturated mono-, di- and triglycerides.

For example, Maisine® CC is an exemplarily modified maize oil. It is obtained by alcoholysis of maize oil and a subsequent winterization of maize oil. The product comprises mono-, di-, and triglycerides, wherein the monoester fraction is comprised from 32% to 52% (w/w), the diester fraction is comprised from 40% to 60% (w/w), and the triester fraction is comprised from 5% to 20% (w/w), based on the total amount of mono-, di-, and triglycerides.

The pharmaceutical composition may comprise sesame seed oil, soya oil, sunflower oil, thistle oil, and modified maize oil, wherein the modification is obtained by alcoholysis, preferably with glycerol, propylene glycol, or low molecular polyethylene glycol.

The pharmaceutical composition can comprise sunflower oil.

The pharmaceutical composition can comprise soya oil.

The pharmaceutical composition can comprise modified maize oil.

The pharmaceutical composition can comprise mixture of modified and unmodified oils.

The pharmaceutical composition can comprise fish oil. The fish oil can be cod-liver oil, salmon oil, or a mixture thereof.

Fish oil may be obtained from fish of families such as Engraulidae, Carangidae, Clupeidae, Osmeridae, Scombridae (except the genera Thunnus and Sarda), and Ammodytidae (type I), or from the genera Thunnus and Sarda with the family Scombridae (type II). The fish oil may comprise omega-3 acids such as alpha-linolenic acid (C18:3 n-3), moroctic acid (C18:4 n-3), eicosatetraenoic acid (C20:4 n-3), timnodonic (eicosapentaenoic) acid (C20:5 n-3; EPA), heneicosapentaenoic acid (C21:5 n-3), clupanodonic acid (C22:5 n-3), and cervonic (docosahexaenoic) acid (C22:6 n-3; DHA). Preferably, the fish oil obtained from type I comprises at least a total of omega-3 acids of 28% (w/w), expressed as triglycerides. The fish oil obtained from type I comprises at least 13% (w/w) of EPA and at least 9% (w/w) of DHA, expressed as triglycerides. Preferably, the fish oil obtained from type II comprises at least a total of omega-3 acids of 28% (w/w), expressed as triglycerides. The fish oil obtained from type II may comprise 4% to 12% (w/w) of EPA and at least 20% (w/w) of DHA, expressed as triglycerides.

Cod-liver oil may be obtained from the fresh livers of cod, Gadus morhua L. and other species of Gadidae, wherein solid substances being removed by cooling and filtering. The cod-liver oil may comprise omega-3 acids such as alpha-linolenic acid (C18:3 n-3), moroctic acid (C18:4 n-3), eicosatetraenoic acid (C20:4 n-3), timnodonic (eicosapentaenoic) acid (C20:5 n-3; EPA), heneicosapentaenoic acid (C21:5 n-3), clupanodonic acid (C22:5 n-3), and cervonic (docosahexaenoic) acid (C22:6 n-3; DHA). Preferably, the cod-liver oil comprises EPA and DHA from 10% to 28% (w/w), expressed as triglycerides. Cod-liver oil may further comprise 3% to 11% (w/w) of linoleic acid, based on the on the total amount of fatty acids.

Salmon oil may be obtained from Salmo salar. The positional distribution (β(2)-acyl) is 60 to 70% for cervonic (docosahexaenoic) acid (C22:6 n-3; DHA), 25% to 35% (w/w) for timnodonic (eicosapentaenoic) acid (C20:5 n-3; EPA), and 40% to 55% (w/w) for moroctic acid (C18:4 n-3). Preferably, the salmon oil comprises EPA and DHA from 10% to 28% (w/w), expressed as triglycerides.

The pharmaceutical composition can comprise sunflower oil and fish oil.

The pharmaceutical composition can comprise modified maize oil and fish oil.

For example, the pharmaceutical compositions described herein can comprise
(A) a hypoxia-inducible factor prolyl hydroxylase inhibitor described herein in an amount of from 0.1% to 20%, preferably from 0.5% to 10% (w/w);
(B) an oil in an amount of from 50% to 99.8%, preferably from 70% to 98.97% (w/w);
(C) optionally a fish oil in an amount of from 0.01% to 5%, preferably from 0.01% to 1.5% (w/w);
(D) optionally a thickener in an amount of from 0.1% to 10%, preferably from 0.5% to 5% (w/w);
(E) optionally an antioxidant in an amount of from 0.01% to 2%, preferably from 0.01% to 1.5% (w/w), and
(F) optionally a preservative in an amount of from 0.01% to 2%, preferably from 0.01% to 1.5% (w/w).

The pharmaceutical composition may not comprise a flavorant. For example, the pharmaceutical composition may not comprise a flavoring agent. The pharmaceutical composition may not comprise a vanilla flavoring agent, anise, honey flavoring agent, or a combination thereof.

### Methods of Making Pharmaceutical Compositions

Methods of preparing the pharmaceutical compositions described herein are manufactured in a manner that is known, including conventional mixing, dissolving, or lyophilizing processes. Thus, liquid pharmaceutical preparations can be obtained by combining the active compounds with solid excipients, optionally grinding the resulting mixture and processing the mixture of granules, after adding suitable auxiliaries, if desired or necessary.

Although preferred embodiments have been depicted and described in detail herein, it will be apparent to those skilled in the relevant art that various modifications, additions, substitutions, can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Preparation and Analysis of Oily Suspension

Oily suspensions of molidustat sodium were prepared by combining the ingredients in the specified amounts as shown in Table 1 below.

TABLE 1
Oily suspension formulations

| | Formulation Type | |
|---|---|---|
| | Oily suspension | Oily suspension w/non- |
| | w/micronized active | micronized active |
| Ingredient | (g/100 ml) | (g/100 ml) |
| molidustat sodium (micronized) | 2.50 | |
| molidustat sodium (non-micronized) | | 2.50 |
| butylhydroxytoluene | 0.12 | 0.12 |
| sorbic acid | 0.08 | 0.08 |
| glycerol dibehenate | 1.00 | 1.00 |
| fish oil | 0.10 | 0.10 |
| sunflower oil, refined | q.s. to 93 | q.s. to 93 |

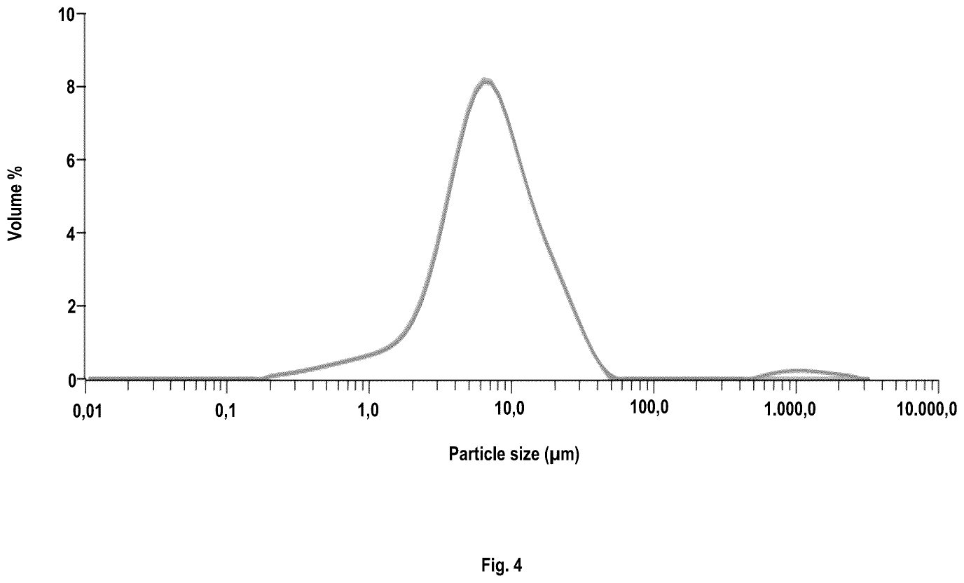

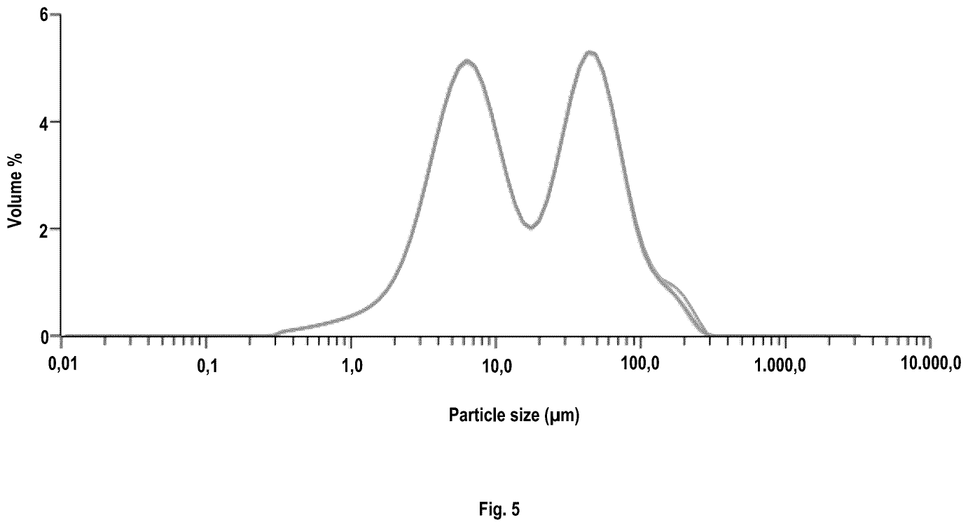

The formulations were analyzed to evaluate active ingredient particle size impact on particle size distribution (PSD) of the finished dose. A Malvern laser diffraction device was used to measure particle sizes in the suspension. Mean particle size measurements are provided in the Table 2 below for each formulation. FIG. 4 and FIG. 5 present particle size distribution histograms of each formulation.

TABLE 2
Particle size distribution of suspension particles

| Formulation | | | |
|---|---|---|---|
| Type | Dx (10) [μm] | Dx (50) [μm] | Dx (90) [μm] |
| non-micronized | 3.23 | 14.7 | 75.2 |
| micronized | 1.7 | 4.7 | 13.3 |

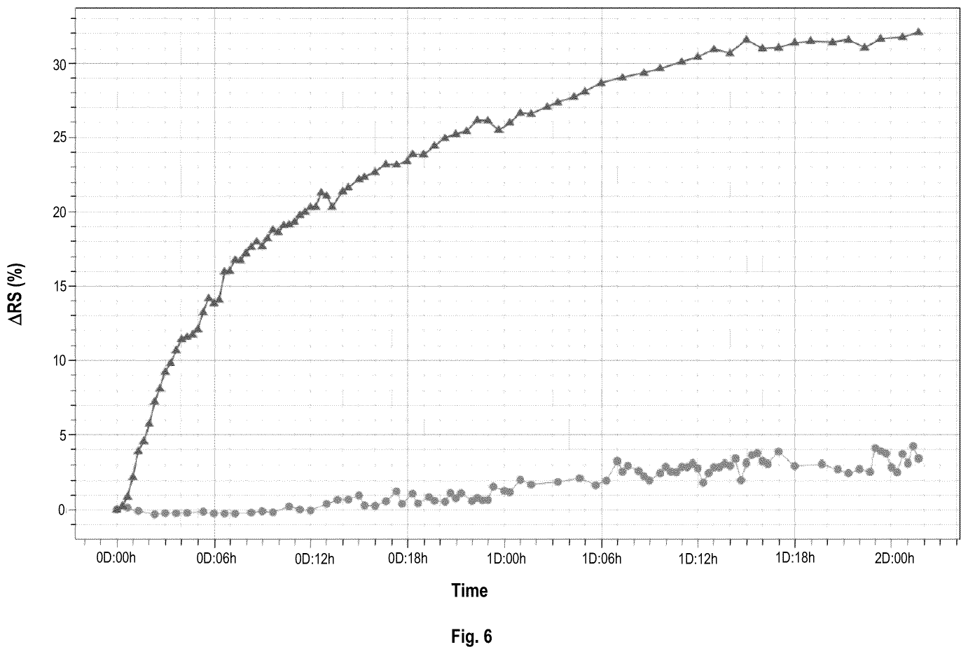

A sedimentation analysis comparing the micronized formulation with the non-micronized formulation was conducted using a TURBISCAN device (optical principles, light transmission & backscattering). The results of the analysis are shown in FIG. 6. The results show that sedimentation of the non-micronized formulation starts within one hour after (re)-homogenization whereas the micronized formulation stays stable for at least six hours and only shows slight sedimentation within 48 hours. The results of this experiment demonstrate that the micronized formulation provides for superior quality control for homogeneity, (re)-suspendability and a slow sedimentation which is crucial for the later administration by the user.

### Example 2

### Plasma Pharmacokinetic Study After Single Oral Administration

The study was a plasma pharmacokinetic study designed to develop the plasma pharmacokinetic profile of molidustat after single oral (po) administration of its sodium salt (micronized) formulated in accordance with Example 1 to fasted and fed cats. Four adult, healthy, female European Shorthaired cats were selected for the study. On the day prior to treatment feed was withdrawn to ensure a fasted prandial state for the oral treatment.

The pharmacokinetic profile of the HIF-PHI was determined in plasma of 3 adult, female, healthy European Shorthaired cats with a mean body weight of 3.5 kg (StD=0.41 kg). One animal was removed from the study due to difficult handling of the animal.

The animals were treated in a fasted prandial state with the test item at a single oral dose rate of 3.0 mg/kg. After a wash out period of 14 days the animals were treated with the test item at the same dose rate and same administration route, but in a fed prandial state. Frequent blood samplings at fixed intervals were performed over a period of 48 hours after each treatment.

The plasma samples were analyzed with regard to their concentrations of molidustat and its metabolite by HPLC using a tandem mass spectrometric detector (AB Sciex API 4000).

From the individual plasma concentration-time-profiles obtained pharmacokinetics (PK) were derived using non-compartmental analyses. Mean plasma pharmacokinetics of molidustat is presented in Table 3.

TABLE 3
Mean plasma pharmacokinetics of molidustat in cats

| Route of | Cₘₐₓ² | Tₘₐₓ³ | t_{1//2}² | AUCᵢₙₜ² | AUCₗₐₛₜ² | MRTᵢₙₜ² | Cl_{b}² | V_{z}² |
|---|---|---|---|---|---|---|---|---|
| Administration | (mg/L) | (h) | (h) | (mg*h/L) | (mg*h/L) | (h) | (L/h/kg) | (L/kg) |
| Oral to fasted | 3.13 | 0.67 | 7.28 | 8.42 | 8.41 | 3.7 | 0.36 | 3.74 |
| cats¹ | | | | | | | | |
| Oral to fed | 0.93 | 1.00 | 6.69 | 6.30 | 6.28 | 7.0 | 0.48 | 4.59 |
| cats¹ | | | | | | | | |

¹Dose applied orally: 3.0 mg molidustat sodium per kg
²Given as geometric mean
³Given as median

Peak and extent of plasma exposure of molidustat was higher if administered orally as sodium salt to cats at fasted prandial state compared to fed prandial state. Metabolized molidustat amounted to approximately 60% in total exposure independent of prandial state. The compounds were clinically well tolerated without any clinical signs of intolerance after two oral treatments at a dose rate of 3.0 mg/kg in cats. Erythropoietin (EPO) concentrations were found highly increased at 6 hours after both treatments, i.e., oral administration to fed or fasted cats. The inventors found that oil administration of molidustat in an oily suspension increased EPO levels rapidly, without the need for intravenous administration.

### Example 3

### Pharmacodynamic Effects on Hematologic Parameters

This study evaluated the effects of molidustat on red blood cell parameters in healthy, adult cats, if administered daily via the oral route at different dosages and using different formulations of molidustat sodium. The hematocrit, erythropoietin (EPO) and molidustat plasma concentrations during the treatment phase were examined, with hematocrit representing the main outcome parameter.

The study had 2 phases, the treatment phase in which increase in hematocrit levels were to be achieved and after cessation of treatment an observation phase, in which hematocrit levels decreased to levels comparable to placebo and within the reference range. An overview of the study design is shown in the Table 4 below.

TABLE 4
Overview on study design, dosing regimen and blood sampling in different study groups.

| | | | | Target dose | | | | | |
|---|---|---|---|---|---|---|---|---|---|
| | | Treatment | | (mg/ | (Ml/ | Days of blood sampling for | | | |
| Study | | period | IVP | kg | kg | | Clinical | | |
| Group | N | (SD) | (suspension) | BW) | BW) | Hematology | chemistry | Molidustat | EPO |
| 1 | 6 | 0-23 | Placebo oily | 0 | 0.1 | −14, −7, 0, 7, | −14, 12⁴, | 0¹, 1, 71, | 0², 1, 7², 8, |
| 2 | 6 | 0-23 | Molidustat | 5 | | 14, 214, 28, | 23³, 97 | 8, 23^{1, 3}, | 23^{2, 3}, 24³ |
| | | | sodium 5% oily | | | 35, 42, 49, | | 24³ | |
| 3 | 5 | 0-15 | Molidustat | 10 | | 56, 70, 84, 98 | | | |
| | | | sodium 10% | | | | | | |
| | | | oily | | | | | | |
| 4 | 5 | 0-15 | Molidustat | 10 | | | | | |
| | | | sodium 10% | | | | | | |
| | | | aqueous | | | | | | |

¹pre-treatment & 2 hours post-treatment,
²pre-treatment & 6 hours post-treatment,
³groups 1 & 2 only,
⁴groups 3 & 4 only.
BW = body weight,
EPO = erythropoietin,
IVP = investigational veterinary product,
No. = number.

Twenty-two adult cats were randomized to three treatment groups and one control group with 5 and 6 cats/group respectively (3 (2) male/3 female). After a baseline phase of two weeks the cats were orally treated in fasted condition once daily. Four different IVP suspensions were administered, placebo oily suspension vehicle, molidustat sodium (micronized) 5% oily suspension, molidustat sodium (micronized) 10% oily suspension, and molidustat sodium (micronized) 10% aqueous suspension. The oily suspensions were in accordance with the oily suspension described in Example 1.

To keep the application volume per kg bodyweight (BW) consistent for each animal the oily suspension of molidustat was available at two different concentrations (5% and 10%), so that each cat was treated with 0.1 mL/kg BW of the respective formulation.

For evaluation of the hematological parameters blood samples were taken at regular intervals before, during and after the treatment phase. Additionally, plasma samples were collected at study day (SD) 0, SD 7 and SD 23 before and after treatment and analyzed for their concentration of EPO and molidustat.

All animals administered molidustat sodium showed an increase in hematocrit above the reference range for up to 42 days after treatment. Hematocrit levels fulfilled criteria for cessation of treatment due to an exaggerated pharmacodynamic effect already on SD 14 in most cats of groups 3 and 4 and on SD 21 in cats of group 2. As a consequence, treatments were omitted in all cats of the respective groups.

Statistical analysis applied to data of the treatment phase resulted in significantly higher mean hematocrit plasma levels in groups treated with 10 mg/kg already at SD 7 and for the 5 mg/kg group at SD 14 compared to placebo. Mean hematocrit levels remained significantly higher for the entire treatment period in all groups. It is noteworthy, that similar results were also seen for mean hemoglobin levels.

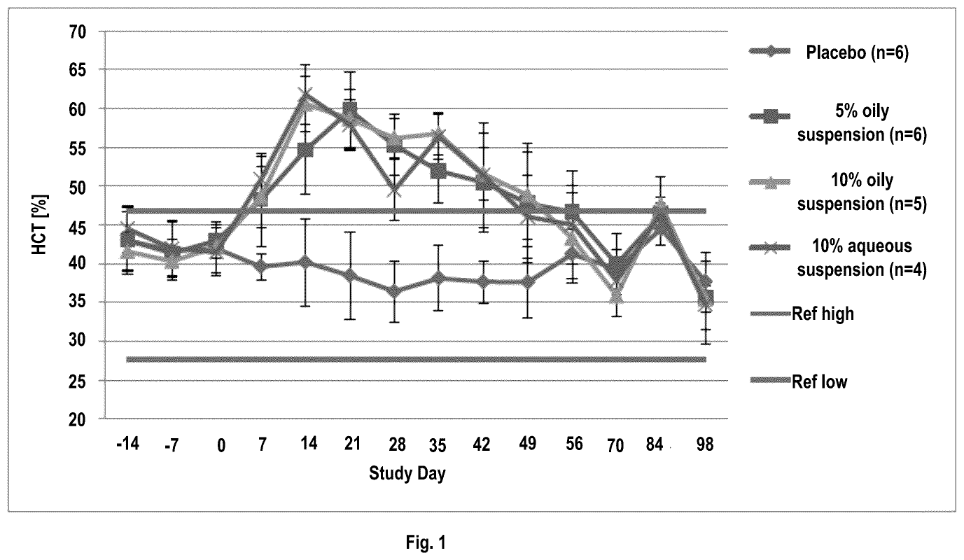

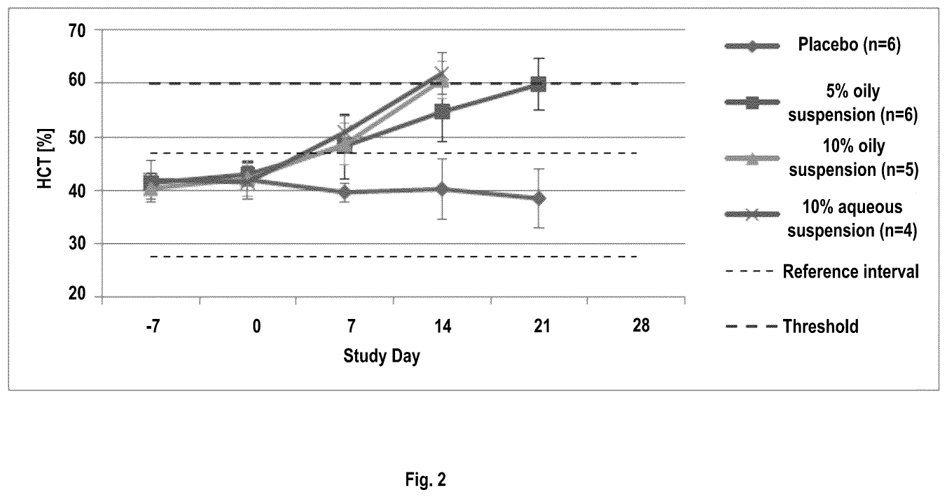

Mean hematocrit levels in molidustat sodium treated groups returned to levels within reference range on SD 56 and were comparable to those of the placebo group from SD 70 onwards. See FIG. 1 and FIG. 2.

EPO concentrations were highest in group 4 cats 6 hours post treatment on SD 0, highest in group 3 cats 6 hours post-treatment on SD 7, coinciding with analysis of molidustat plasma concentrations. EPO concentrations (at 6 hours post treatment) significantly differed in all molidustat sodium treated groups compared to the placebo group at all study days measured.

The EPO concentrations in the plasma of cats receiving placebo (group 1) remained at endogenous levels throughout the study.

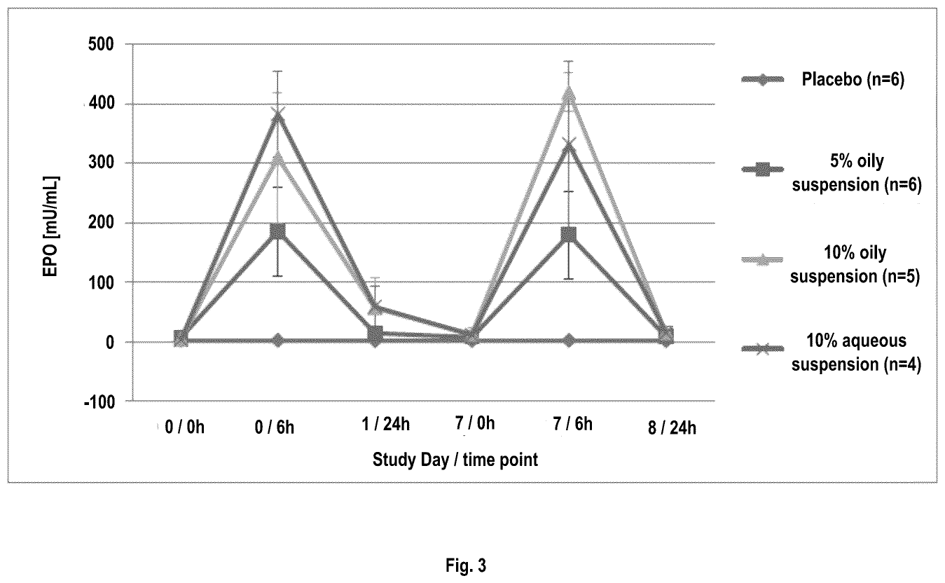

Six hours after administration on SDs 0 and 7, the mean EPO concentration peaked in all treated groups. On SD 0 six hours after administration the EPO concentration was highest in group 4, then group 3 and then group 2 cats. However, on SD 7, the mean EPO concentration was highest in group 3 then group 4, then group 2 cats, six hours after administration. At 24 hours post-treatment on SD 1, the mean EPO concentration was approximately 55 mU/mL in groups 3 and 4, and almost back to pre-treatment values in group 2. At 24 hours post-treatment on SD 8, the concentration of EPO in the plasma was almost back to pre-treatment values for all groups. See FIG. 3.

On SDs 0 and 7, the EPO concentration for all treated groups significantly differed to the placebo group. On SD 23 the EPO concentration in group 2 (remaining group treated) significantly differed to the placebo group as well.

Treatments with molidustat sodium were very well tolerated and safe in all study groups. Treatments had no impact on the cats' body weight. None of the findings observed during the physical examinations or general health observations were rated as having a negative impact on the outcome of the study. The most frequently observed findings were vomiting of small amounts of fluid. This adverse event related to treatment (within 4 hours after treatment) were observed across all groups: they were seen in groups 1-3 at a mild rate below 10% (group 1: 0.7%, group 2:3.5% and group 3:8.8%), and at a moderate rate of 20% in group 4, indicating that administration of the aqueous suspension was less tolerated than those of the oily suspensions.

### Example 4

### Efficacy in Cats with Anemia Associated with Chronic Kidney Disease (CKD)

The objective of this study was to evaluate the safety and efficacy of molidustat to manage anemia associated with chronic kidney disease (CKD) in cats.

The study was a multi-site, globally randomized, masked, placebo (vehicle) controlled field study, designed to evaluate the efficacy and in-use safety of molidustat (micronized) oral suspension in accordance with Example 1 in cats with anemia associated with CKD.

This study was conducted using a single protocol in the United States (US, n=23 clinics) and Europe (EU, n=11 clinics) over a 10 month period. On study day (SD) −7 (±2 days), blood was collected for serum chemistry and hematology from cats that qualified for enrollment. After meeting enrollment criteria, cats were orally treated once daily for at least 28 consecutive days (SD 0 to SD 27) with either the control product (CP [vehicle]) or molidustat. The molidustat was administered orally at a dose of 5 mg/kg body weight (bw) (0.2 mL/kg bw). Blood was collected to assess hematological parameters on SDs 0, 7, 14, 21 and 28 (±2 days for all time points except SD 0). Blood was also collected for evaluating serum chemistry on SD 28 (±2 days), except creatinine. Hematocrit (HCT) and packed cell volumes (PCV, performed at each clinic) were the primary hematological parameters of interest. Individual animal treatment success was evaluated up to and including SD 28±2 using three different criteria, as well as using only the data on SD 28.

A total of 65 cats were screened at 11 US and 9 EU sites. Twenty-three (23) cats (13 cases from 9 sites in the US and 10 cases from 9 sites in the EU), with nearly equally distributed genders, mostly domestic shorthair breed, ranging in age from 4 to 17 years, and having initial body weights between 2 and 6 kilograms, were randomized and administered one of the two treatments. A total of 21 cats were included in the 28-day efficacy phase.

The clinical conclusions from analyzing the HCT and PCV results were nearly identical. The anemic CKD control group (CP, n=6) average weekly responses varied as expected and the study participation declined throughout the study. Only three (3) CP cats completed the 28-day study. Four of the six CP-treated cats did not show any relevant increases in both of these parameters. The treatment group average weekly responses ranged from increases of 1.65% to 3.91%, as compared to the average baseline. Compared to the CP group, HCT and PCV results were significantly greater on SD 21 (p-values<0.01). The increases observed on SD 28 approached statistical significance (p<0.061), mainly due to the limited number of CP cats providing data on this study day. Compared to each treatment group's baseline (baseline-controlled analyses), both SD 21 and SD 28 HCT were significantly different than the average baseline of 3.91% and 3.68%, respectively. In addition, PCV values at SD 21 and 28 compared to each treatment's group baseline (baseline-controlled analyses) were significantly different than the average baseline of 3.46% and 3.89%, respectively. Also, SD 14 HCT increase averaged 1.87% relative to baseline (approaching significance, p=0.0643) and SD 14 PCV increase averaged 2.21% relative to baseline (p=0.0167). In contrast, no significant HCT or PCV increases relative to baseline were observed in the CP group.

The proportion of each treatment group's individual animal treatment success, based on HCT values and depending upon the success criteria, ranged from 40% to 60% for the IVP treatment group and was 16.7% for the CP group. Treatment success based on PCV results ranged from 33% to 67% for the treatment group and 33% for the CP group. Although approximately 20%-40% improvement was observed between the two groups, the limited sample size (n=15 and n=6, for the IVP and CP groups, respectively) resulted in no statistically significant differences.

There were 3 cats from the US and 5 cats from the EU in the continuation phase. Two cats received 5 mg/kg bw throughout the continuation phase; 5 cats were intermittently treated with 2.5 mg/kg bw and 1 cat was treated with 5 mg/kg bw until SD 49. In general, the HCT and PCV levels in all 8 cats were maintained or increased throughout the continuation phase except for one cat in the EU region (GA03) that had a lower PCV on SD 77. However, its PCV increased to 26% on SD 84. The results from the cats in the continuation phase indicated that by intermittent treatment and/or by adjusting the dose of molidustat, acceptable HCT/PCV levels can be maintained or achieved with no treatment related adverse events.

No treatment related adverse events were noticed when cats were administered molidustat or with other concomitant medications that were used in this study to treat the symptoms of CKD. Molidustat was administered once daily for 28 days at 5 mg/kg body weight significantly increased the PCV/HCT on SD 21 and SD 28 in comparison to SD 0 in anemic cats. The treatment was well tolerated, and no treatment related adverse events were noticed when molidustat was administered alone or in combination with other medications that were used to treat symptoms of CKD.

### Example 5

### Plasma Pharmacokinetics of Molidustat After Repeated Oral Dosing of 2.5% (M/V) Molidustat Oily Suspension

The plasma pharmacokinetic profile of the test item (molidustat sodium (micronized) oily suspension 2.5% (m/v) in accordance with Example 1) was determined after repeated once daily oral dosing at a target dose rate of 5 mg molidustat sodium per kg bw to healthy adult cats.

Following a single group design, eight healthy, young adult cats (4 neutered males, 4 spayed females) were included in the study. The animals were between 14 and 15.5 months old and weighed between 3.35 and 4.95 kg at start of the in-life phase.

The dose rate administered was 4.8 mg molidustat sodium per kg. Dosing was once daily on 6 consecutive days keeping an interval of 24 h. Dose rate calculation was based on the body weights determined on SD-1 for all administrations.

Thirty-one blood samples were drawn per animal following a pre-determined schedule deemed suitable to describe the pharmacokinetic profile of the active substance contained in the test item after repeated dosing to cats.

Concentrations of the active substance molidustat and its major metabolite in plasma were analyzed by High Performance Liquid Chromatography/Tandem Mass Spectrometry. The lower limit of quantitation was 5 μg/L for each substance.

Pharmacokinetic evaluation of plasma concentration data was based on the observed data using non-compartmental methods and comprised all PK-parameters to adequately describe the absorption, distribution and elimination phase of the active substance and its metabolite after repeated dosing with the test item. Table 5 presents a summary of mean plasma pharmacokinetics of molidustat and its metabolite, which are displayed as derived after repeated once daily oral dosing of the test item.

TABLE 5
Summary of mean plasma pharmacokinetics of molidustat and its metabolite

| | | Cₘₐₓ | Tₘₐₓ | t_{1/2} | AUCₜₐᵤ |
|---|---|---|---|---|---|
| Treatment | Active Substance | (mg/L) | (h) | (h) | (mg*h/L) |
| First | molidustat | 4.46 (14.1%) | 0.64 (60.1%) | 4.2 (12.2%) | 13.26 (21.7%) |
| Dose | molidustat | 1.44 (13.1%) | 2.45 (21.9%) | 4.3 (12.0%) | 8.13 (18.2%) |
| | metabolite | | | | |
| Last (6^{th}) | molidustat | 4.10 (21.0%) | 0.94 (0.5%) | 6.5 (39.1%) | 13.34 (19.1%) |
| Dose | molidustat | 1.49 (16.1%) | 2.50 (0.4%) | 7.5 (41.9%) | 8.85 (19.9%) |
| | metabolite | | | | |

Molidustat = molidustat sodium oily suspension 2.5% (m/v) oral administration
Dose rate applied was 4.8 mg molidustat sodium per kg once daily;
¹Mean values given as geometric mean and coefficient of variation

Under the conditions of the actual study derived plasma pharmacokinetics were characterized by a low inter-individual variation. Six repeated once daily doses of the test item at an actual dose rate of 4.8 mg molidustat sodium per kg bw resulted in steady-state plasma pharmacokinetics.

No clinically relevant accumulation occurred after repeated once daily dosing at the intended therapeutic dose rate with a calculated accumulation index of 1.1.

No changes or alterations in the derived plasma pharmacokinetics occurred after repeated dosing comparing the pharmacokinetics after the first and the last dose, indicating that repeated once daily dosing at a rate of 5 mg molidustat sodium per kg does not affect metabolism or elimination pattern of molidustat in cats.

### Example 6

### Bioavailability of Molidustat

In this study, the plasma pharmacokinetic properties of item (molidustat sodium (micronized) oily suspension 2.5% (m/v) in accordance with Example 1) were determined with special regard to oral bioavailability and dose proportionality. The test item was applied as single oral dose at the expected target dose rate of 5 mg molidustat sodium per kg bw, at 2.5 mg/kg and at 10 mg/kg to healthy adult cats. A single intravenous bolus dose of 5.0 mg/kg was applied for comparison using a suitable reference item (molidustat sodium aqueous solution 2.0% (m/v)).

Following a mixed serial/cross-over design, 16 healthy, adult cats (8 neutered males, 8 spayed females) were included in the study and allocated to 4 study groups of 8 animals each. The animals were between 13.7 and 14.7 months old and weighed between 3.3 and 6.0 kg at start of the in-life phase.

The dose rates administered were at means of 2.42 mg/kg, 4.79, and 9.54 mg molidustat sodium per kg for the 0.5×, 1×, 2× dose groups, respectively. The reference item was dosed at an actual mean dose rate of 5.15 mg/kg. Dose rate calculations were based on the body weights determined prior to each study period.

Fifteen blood samples were drawn per animal after oral dosing (16 samples/animal after intravenous dosing) following a pre-determined schedule deemed suitable to describe the pharmacokinetic profile of the active substance contained in the test and reference item after single oral or intravenous dosing to cats.

Concentrations of the active substance molidustat and its major in plasma were analyzed by High Performance Liquid Chromatography/Tandem Mass Spectrometry in accordance with method 01469 (Krebber et al., Analytical method for the determination of BAY 85-3934 and its metabolite BAY 116-3348 in plasma by LC-MS/MS, BAG-CS report MR-15/109). The lower limit of quantitation was 5 μg/L for each substance.

Pharmacokinetic evaluation of plasma concentration data was based on the observed data using non-compartmental methods and comprised all PK-parameters to adequately describe the absorption, distribution and elimination phase of the active substance and its metabolite after oral or intravenous dosing with the test or reference item. For pharmacokinetic evaluation actual individual dose rates were used to account for the small differences between target and actual dose rates. All animals finished the in-life phase according to schedule. The animals showed a good general health during the entire in-life phase. A summary of the mean plasma pharmacokinetics is presented in Table 6.

TABLE 6
Selected mean plasma pharmacokinetics derived for the test item

| | Active | Cₘₐₓ | Tₘₐₓ | t_{1/2} | AUCₜₐᵤ |
|---|---|---|---|---|---|
| Dose Rate | Substance | (mg/L) | (h) | (h) | (mg*h/L) |
| 2.5 mg/kg | molidustat | 2.7 (29.0%) | 0.5 (81.8%) | 5.0 (21.1%) | 5.60 (11.5%) |
| | molidustat | 0.82 (17.6%) | 2.4 (21.9%) | 4.7 (15.7%) | 4.39 (19.9%) |
| | metabolite | | | | |
| 5.0 mg/kg | molidustat | 3.83 (13.0%) | 1.0 (21.9%) | 4.6 (14.9%) | 12.71 (21.7%) |
| | molidustat | 1.71 (18.1%) | 3.0 (0%) | 7.5 (28.2%) | 9.51 (18.8%) |
| | metabolite | | | | |
| 10 mg/kg | molidustat | 6.73 (38.9%) | 1.0 (77.6%) | 7.1 (62.6%) | 23.51 (15.8%) |
| | molidustat | 2.18 (18.5%) | 2.9 (14.4%) | 6.4 (24.7%) | 14.50 (18.9%) |
| | metabolite | | | | |

molidustat = molidustat sodium oily suspension 2.5% (m/v) oral administration
¹Mean values given as geometric mean and coefficient of variation
²Actual dose rates applied were 2.46 mg, 4.79 mg, and 9.54 mg molidustat sodium per kg, single dose

After a single oral administration of the test item molidustat sodium oily suspension 2.5% (m/v) at a dose rate of 5 mg molidustat sodium per kg to cats, oral bioavailability was high with a geometric mean of 82%, ranging from 57% to 110%.

Oral administration of the test item at dose rates of 2.5 and 10 mg molidustat sodium per kg, corresponding to 0.5× to 2× the targeted therapeutic dose rate resulted in dose proportional extent of plasma exposure. The rate of plasma exposure (Cmax) was slightly under-proportional.

After a single intravenous bolus dose of 5 mg molidustat sodium per kg to cats, mean molidustat plasma exposure was 16.35 mg*h/L. Molidustat was distributed in the body with a volume of 2.69 L/kg. Plasma clearance was 0.31 L/h/kg. Molidustat was eliminated with a half-life of 5.91 h.

The metabolite was built at an extent of 8.71 mg*h/L (conversion rate of 53%). Plasma clearance was 0.59 L/h/kg and elimination half-life was 5.13 h. Inter-animal variation was low. Comparison between male and female animals showed a slightly higher plasma clearance (0.33 vs 0.30 L/h/kg) and volume of distribution (2.96 vs 2.36 L/kg) in females resulting in slightly higher total plasma exposure for both, parent and metabolite.

In conclusion, under the conditions of the actual study, the test item molidustat sodium oily suspension 2.5% (m/v) showed an unexpected high oral bioavailability (83%) at the targeted therapeutic dose rate of 5 mg molidustat sodium per kg in cats. Plasma exposure was dose proportional over the tested dose range of 0.5× to 2× (2.5 to 10 mg/kg). No relevant gender differences were present.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A pharmaceutical composition comprising a hypoxia-inducible factor prolyl hydroxylase inhibitor and an oil.

2. The pharmaceutical composition of claim 1, wherein the hypoxia-inducible factor prolyl hydroxylase inhibitor comprises a compound of Formula (I) 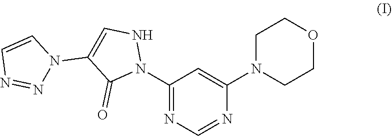 [SMILES: O=c1c(-n2ccnn2)c[nH]n1-c1cc(N2CCOCC2)ncn1]
or a salt, stereoisomer, tautomer, or N-oxide thereof.

3. The pharmaceutical composition of claim 1, wherein the compound of Formula I is in the form a salt having the Formula (II) 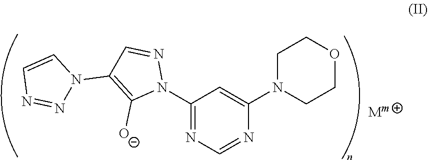
wherein M is lithium, sodium, potassium, calcium, magnesium, barium, manganese, copper, silver, zinc, iron, ammonium, or a substituted ammonium in which one to four of the hydrogen atoms are replaced by C₁-C₄-alkyl;
m denotes the respective positive charge of the cation, being 1, 2, or 3, preferably 1; and n denotes the respective stoichiometric amount of the counter anion and is 1, 2, or 3,
preferably 1; wherein n equals m so that the salt having the formula (II) is uncharged.

4. The pharmaceutical composition of claim 1, wherein the hypoxia-inducible factor prolyl hydroxylase inhibitor comprises a compound of Formula (IIA):

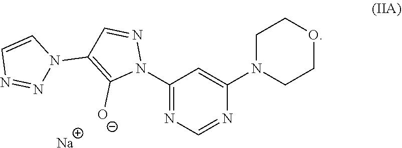 [SMILES: [O-]c1c(-n2ccnn2)cnn1-c1cc(N2CCOCC2)ncn1 ; [Na+]]

5. The pharmaceutical composition of claim 1, wherein the hypoxia-inducible factor prolyl hydroxylase inhibitor comprises a sodium salt.

6. The pharmaceutical composition of claim 1, wherein the hypoxia-inducible factor prolyl hydroxylase inhibitor comprises or consists of molidustat.

7. The pharmaceutical composition of claim 1, wherein the hypoxia-inducible factor prolyl hydroxylase inhibitor comprises or consists of molidustat sodium.

8. The pharmaceutical composition of claim 1, wherein the composition comprises micronized particles comprising the hypoxia-inducible factor prolyl hydroxylase inhibitor.

9. The pharmaceutical composition of claim 8, wherein the micronized particles are characterized by a D90 that is about 70 mm or less, about 60 mm or less, about 50 mm or less, about 40 mm or less, or about 30 mm or less.

10-13. (canceled)

14. The pharmaceutical composition of claim 1, wherein the hypoxia-inducible factor prolyl hydroxylase inhibitor concentration is from about 1.0% and 20% (m/v).

15-59. (canceled)

60. A method for increasing erythropoietin comprising administering the pharmaceutical composition of claim 1 to a subject in need thereof.

61. A method for treating anemia comprising administering the pharmaceutical composition of claim 1 to a subject in need thereof.

62. The method of claim 61, wherein the anemia is non-regenerative anemia.

63. The method of claim 61, wherein the anemia is iron-deficiency anemia, pernicious anemia, aplastic anemia, hemolytic anemia, chemotherapy-induced anemia (CIA), or immune mediated hemolytic anemia (IMHA).

64. The method of claim 61, wherein the pharmaceutical composition is administered once daily.

65-67. (canceled)

68. The method of claim 61, wherein the subject in need thereof is a mammal.

69. The method of claim 61, wherein the subject is a cat.

70. The method of claim 61, wherein the composition is administered orally.

71. The method of claim 61, wherein the composition is administered at sufficient dose to provide a maximum plasma concentration (Cmax) of the hypoxia-inducible factor prolyl hydroxylase inhibitor of about 0.5 mg/L or greater, about 1 mg/L or greater, 1.5 mg/l or greater, 2 mg/L or greater, 2.5 mg/L or greater, 3 mg/L or greater, or from about 0.5 mg/L to about 5 mg/L.
